SEQUENCE LISTING (11 sequences)

SEQ ID NO: 1 - protein, 657 aa, synthetic construct
MGHHHHHHSSGHIEGRHMASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDFEDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 2 - protein, 553 aa, synthetic construct
VYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 3 - protein, 680 aa, Fusarium graminearum
MKHFLSLALCFSSINAVAVTVPHKSGGTGSPEGSLQFLSLRASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQ

SEQ ID NO: 4 - protein, 351 aa, Bacillus sp.
MKNMPENHNPQANAWTAEFPPEMSYVVFAQIGIQSKSLDHAAEHLGMMKKSFDLRTGPKHVDRALHQGADGYQDSIFLAYWDEPETFKSWVADPEVQKWWSGKKIDENSPIGYWSEVTTIPIDHFETLHSGENYDNGVSHFVPIKHTEVHEYWGAMRDRMPVSASSDLESPLGLQLPEPIVRESFGKRLKVTAPDNICLIRTAQNWSKCGSGERETYIGLVEPTLIKANTFLRENASETGCISSKLVYEQTHDGEIVDKSCVIGYYLSMGHLERWTHDHPTHKAIYGTFYEMLKRHDFKTELALWHEVSVLQSKDIELIYVNCHPSTGFLPFFEVTEIQEPLLKSPSVRIQ

SEQ ID NO: 5 - protein, 364 aa, Bacillus sp.
MKNMPENHNPQANAWTAEFPPEMSYVVFAQIGIQSKSLDHAAEHLGMMKKSFDLRTGPKHVDRALHQGADGYQDSIFLAYWDEPATFKSWVADPEVQKWWSGKKIDENSPIGYWSEVTTIPIDHFETLHSGENYDNGVSHFVPIKHTEVHEYWGAMRDRMPVSASSDLESPLGLQLPEPIVRESFGKRLKVTAPDNICLIRTAQNWSKCGSGERETYIGLVEPTLIKANTFLRENASETGCISSKLVYEQTHDGEIVDKSCVIGYYLSMGHLERWTHDHPTHKAIYGTFYEMLKRHDFKTELALWHEVSVLQSKDIELIYVNCHPSTGFLPFFEVTEIQEPLLKSPSVRIQKLAAALEHHHHHH

SEQ ID NO: 6 - protein, 353 aa, Rhodococcus globerulus
MESAIGEHLQCPRTLTRRVPDTYTPPFPMWVGRADDTLHQVVMGYLGVQFRGEDQRPAALRAMRDIVAGFDLPDGPAHHDLTHHIDNQGYENLIVVGYWKDVSSQHRWSTSPPVSSWWESEDRLSDGLGFFREIVAPRAEQFETLYAFQDDLPGVGAVMDGVSGEINEHGYWGSMRERFPISQTDWMQASGELRVVAGDPAVGGRVVVRGHDNIALIRSGQDWADAEADERSLYLDEILPTLQSGMDFLRDNGPAVGCYSNRFVRNIDIDGNFLDLSYNIGHWASLDQLERWSESHPTHLRIFTTFFRVAEGLSKLRLYHEVSVFDAADQLYEYINCHPGTGMLRDAVITAEH

SEQ ID NO: 7 - protein, 363 aa, Fusarium graminearum
MLRSRFPASHHFTVSVFGCQYHSEAPSVEKTELIGRFDKLIDSAAIHVEHLEQNDVPSKIWMSYWESPQKFKQWWEKDDTASFWASLPDDAGFWRETFSLPATRAMYEGTGKDAYGFGHCGSLIPLTTKTGYWGAYRSRMTPDFEGDTFSSPIPTYADQSVPADKIRPGRVRITDFPDNLCMVVEGQHYADMGEREREYWNENFDGLTKQWVTNVVTAGHEQGMVIARACHGFAGEKKLGATNGPVNGIFPGLDYVHQAQILIWQDISKMEHIGRYDQTHVKLRRDFMKAYGPGGEMEGGDLLLWVDLGILKKDEIDAEYVGCYESTGFLKLDKGQFFKVESTAGSKLPSFFDEPIESKPIEW

SEQ ID NO: 8 - protein, 353 aa, Rhodococcus erythropolis
MESAIGEHLQCPRTLTRRVPDTYTPPFPMWVGRADDALQQVVMGYLGVQFRDEDQRPAALQAMRDIVAGFDLPDGPAHHDLTHHIDNQGYENLIVVGYWKDVSSQHRWSTSTPIASWWESEDRLSDGLGFFREIVAPRAEQFETLYAFQEDLPGVGAVMDGISGEINEHGYWGSMRERFPISQTDWMQASGELRVIAGDPAVGGRVVVRGHDNIALIRSGQDWADAEADERSLYLDEILPTLQSGMDFLRDNGPAVGCYSNRFVRNIDIDGNFLDLSYNIGHWASLDQLERWSESHPTHLRIFTTFFRVAAGLSKLRLYHEVSVFDAADQLYEYINCHPGTGMLRDAVTIAEH

SEQ ID NO: 9 - protein, 352 aa, Pseudomonas chlororaphis
MESAIDTHLKCPRTLSRRVPEEYQPPFPMWVARADEQLQQVVMGYLGVQYRGEAQREAALQAMRHIVSSFSLPDGPQTHDLTHHTDSSGFDNLMVVGYWKDPAAHCRWLRSAEVNDWWTSQDRLGEGLGYFREISAPRAEQFETLYAFQDNLPGVGAVMDSTSGEIEEHGYWGSMRDRFPISQTDWMKPTNELQVVAGDPAKGGRVVIMGHDNIALIRSGQDWADAEAEERSLYLDEILPTLQDGMDFLRDNGQPLGCYSNRFVRNIDLDGNFLDVSYNIGHWRSLEKLERWAESHPTHLRIFVTFFRVAAGLKKLRLYHEVSVSDAKSQVFEYINCHPHTGMLRDAVVAPT

SEQ ID NO: 10 - protein, 345 aa, Bradyrhizobium sp.
MESAIPSHLETARSRHRRVPDDYAPPYPSFVARHKPAVLSVIMAYFGVQYRGEPTAAATEALAQIVARFAADNGPTHWDRAQYVDQAGFTNVVSVAYWDDAARFDAWFEQAREAWTGSGASDGVGRFIEVLRPHVERYETLFSSLGRPEGVAVLADGMSGEVQEHAYWGGMRDRIPLSQTDPMAPNGEPRAIRDGARIRVVAHDNLCLIRSGQDWSDTEDSERKMYLDDVEPVLREGMDFLRDDGGPIGCYANRYMRVVDADGRLTEKSYGQSWWKSLATLERWAESHPTHVRIFGAAMKYLSTLGPSAKLRLYHEVTVAAADEQFFEYLGCHERTGMLGAVQAA

SEQ ID NO: 11 - protein, 352 aa, Pseudomonas sp.
MESAIDTHLKCPRTLSRRVPDEYQPPFAMWMARADEHLEQVVMAYFGVQYRGEAQRAAALQAMRHIVESFSLADGPQTHDLTHHTDNSGFDNLIVVGYWKDPAAHCRWLRSAPVNAWWASEDRLNDGLGYFREISAPRAEQFETLYAFQDNLPGVGAVMDRISGEIEEHGYWGSMRDRFPISQTDWMKPTSELQVIAGDPAKGGRVVVLGHGNLTLIRSGQDWADAEAEERSLYLDEILPTLQDGMDFLRDNGQPLGCYSNRFVRNIDLDGNFLDVSYNIGHWRSVEKLERWTESHPTHLRIFVTFFRVAAGLKKLRLYHEVSVSDAKSQIFGYINCHPQTGMLRDAQVSPA

(19) United States
(12) Patent Application Publication
(10) Pub. No.: US 2026/0201432 A1
(43) Pub. Date: Jul. 16, 2026

(54) BIOBASED SYNTHESIS OF GLUCODIAMINE

(71) Applicant: Solugen, Inc., Houston, TX (US)

(72) Inventors: Agustin Millet, Houston, TX (US); Hans-Joerg Woelk, Houston, TX (US); Toni M. Lee, Missouri City, TX (US); Gaurab Chakrabarti, Houston, TX (US); Sean Hunt, Houston, TX (US)

(73) Assignee: Solugen, Inc., Houston, TX (US)

(21) Appl. No.: 19/138,720

(22) PCT Filed: Dec. 13, 2023
(86) PCT No.: PCT/US2023/083879

§ 371 Date: Jun. 13, 2025

Related U.S. Application Data
(60) Provisional application No. 63/432,369, filed on Dec. 13, 2022.
(60) Provisional application No. 63/439,416, filed on Jan. 17, 2023.

(51) Int. Cl. C12P 13/00

(52) U.S. Cl. CPC C12P 13/001

(57) ABSTRACT

A method of preparing glucodiamine, comprising contacting glucose with an enzyme oxidation catalyst under conditions suitable for the formation of glucodialdose; contacting glucodialdose with a nitrogen-containing compound under conditions suitable for the formation of glucodioxime; and reducing glucodioxime under conditions suitable for the formation of glucodiamine. A method of preparing glucodiamine, comprising contacting glucodioxime with a dehydration catalyst under conditions suitable for the formation of glucodinitrile; and reducing glucodinitrile under conditions suitable for the formation of glucodiamine. A chemoenzymatic method of producing a bio-based amide platform chemical, comprising: contacting glucose with a biocatalyst under conditions suitable for the formation of glucodialdose; contacting glucodialdose with a base under conditions suitable for the formation of glucodioxime; and contacting glucodioxime with a hydrogenation catalyst in the presence of hydrogen under conditions suitable for formation of glucodiamine.

### CROSS-REFERENCE TO RELATED APPLICATIONS

This application is a 35 U.S.C. § 371 national stage application of PCT/US2023/083879 filed Dec. 13, 2023 and entitled “Biobased Synthesis of Glucodiamine,” which claims priority to U.S. Provisional Application No. 63/432,369 filed Dec. 13, 2022 and to U.S. Provisional Application No. 63/439,416 filed Jan. 17, 2023, each entitled “Biobased Synthesis of Glucodiamine,” each of which is hereby incorporated herein by reference in its entirety for all purposes.

### REFERENCE TO SEQUENCE LISTING

The instant application contains a Sequence Listing which has been submitted electronically in XML file format and is hereby incorporated by reference in its entirety. Said XML file, created on Aug. 2, 2025, is named “22PRC018-PCT_3416-14004_Sequence Listing.xml” and is 14.9 kilobytes in size.

### TECHNICAL FIELD

The present disclosure relates generally to compositions and methods for the production of biobased monomers. More particularly, the present disclosure relates to chemoenzymatic methods for the production of nitrogen-containing platform chemicals.

### BRIEF DESCRIPTION OF DRAWINGS

For a detailed description of various exemplary embodiments, reference will now be made to the accompanying drawings in which:

FIG. 1 is a schematic depiction of glucodiamine production from glucose in accordance with the principles described herein.

FIG. 2 is a schematic depiction of glucodiamine production from glucodioxime in accordance with the principles described herein.

FIG. 3 is a bar graph illustrating the specific activity of the indicated GAO mutants on glucose.

FIG. 4 is a bar graph illustrating the specific activity of the indicated GAO mutants on gluconate.

FIG. 5 is a bar graph illustrating the specific activity of GAO-Mut47 and 107 on 0.5% and 2% glucose.

FIG. 6 is a bar graph illustrating the activity of the Mut47 in a Parr reaction system at 11° C. after the indicated reaction times.

FIG. 7A is a bar graph illustrating the specific activity of machine learning mutants compared with GAO-mut47 and GAO-Mut107 controls.

FIG. 7B is a graph illustrating the T50 of machine learning mutants compared with GAO-mut47 and GAO-Mut107 controls.

FIG. 8A is a bar graph illustrating the glucose concentration before and after the first step of a two-step reaction to convert glucose to L-guluronic acid.

FIG. 8B is a graph illustrating glucose, gluconic acid and L-guluronic acid concentration as a function of the reaction time.

FIGS. 8C-8G are High Performance Liquid Chromatography Mass Spectroscopy (HPLC-MS) trace overlays from reaction samples of the two step reaction with appropriate authentic standards at different M/z channel with negative mode.

FIG. 9 is a graph of the amount of reactant consumed (glucodioxime) and product generated (glucodiamine) as a function of time.

FIG. 10 is a high performance liquid chromatography-mass spectra of a product fraction containing glucodiamine that shows the exact mass of the product.

### BACKGROUND

Nitriles are among the most important functional groups in chemistry and are of outstanding importance within the product tree of today's chemical industry. Numerous product segments contain nitriles, ranging from pharmaceuticals and fine chemicals in the high price-low volume segment to the field of polymer building blocks and solvents as low price, but high volume chemicals.

Nylon-6 and Nylon-6,6 constitute around 90% of the total nylon produced daily worldwide, which amounts to approximately 7.0 million tons annually. Industrially, Nylon-6 polymer is typically generated via the ring-opening polymerization of ε-caprolactam by employing cyclohexanone as a major precursor. Similarly, 6-aminohexanoic acid (6-AmHA) can be cyclized into ε-caprolactam by employing suitable ring-cyclizing enzymes and this serves as a valuable alternative starting material to obtain Nylon 6. Nylon 4, 5 and 6 are polyamides that can be used for the engineering of commercial products having superior thermal and mechanical properties such as plastics, tire cords, carpeting, and food packaging materials. However, complete biosynthetic pathways or greener production processes for the synthesis of nitrogen-containing building blocks, and subsequently polyamides, from renewable starting materials are still under development. For example, there is no current method for the synthesis of glucodiamine, also known as diethanolamine glucuronate, which is a green platform molecule that can be used in the production of polyamides.

### SUMMARY

Disclosed herein is a method of preparing glucodiamine, comprising contacting glucose with an enzyme oxidation catalyst under conditions suitable for the formation of glucodialdose; contacting glucodialdose with a nitrogen-containing compound under conditions suitable for the formation of glucodioxime; and reducing glucodioxime under conditions suitable for the formation of glucodiamine.

Also disclosed herein is a method of preparing glucodiamine, comprising contacting glucodioxime with a dehydration catalyst under conditions suitable for the formation of glucodinitrile; and reducing glucodinitrile under conditions suitable for the formation of glucodiamine.

Also disclosed herein is a chemoenzymatic method of producing a bio-based amide platform chemical, comprising contacting glucose with a biocatalyst under conditions suitable for the formation of glucodialdose; contacting glucodialdose with a base under conditions suitable for the formation of glucodioxime; and contacting glucodioxime with a hydrogenation catalyst in the presence of hydrogen under conditions suitable for formation of glucodiamine.

### DETAILED DESCRIPTION

Disclosed herein are novel routes for the synthesis of a primary diamine from a renewable resource. In an aspect, the renewable resource comprises a sugar feedstock, a partially purified sugar feedstock, a purified sugar, or a combination thereof. In an aspect, the renewable resource comprises a biomass.

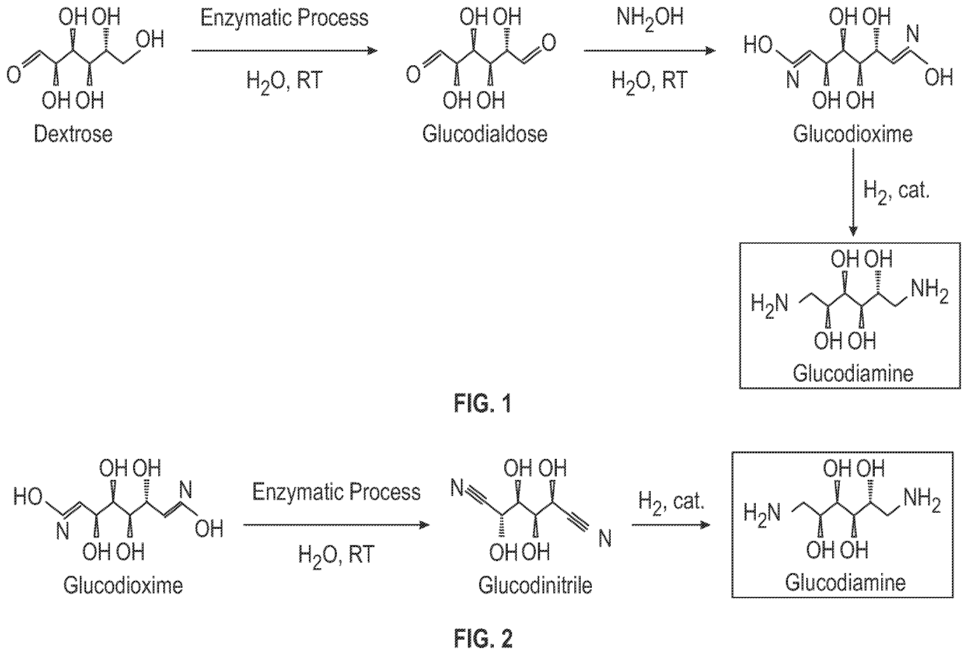

In one or more aspects, a method for the production of glucodiamine from glucose, is depicted schematically in FIG. 1. With reference to FIG. 1, glucose is contacted with an enzyme oxidation catalyst under conditions suitable for production of glucodialdose. For example, the enzyme oxidation catalyst is a copper radical oxidase (CRO).

CROs are non-flavoprotein alcohol oxidoreductases that employ molecular oxygen as a terminal electron acceptor to generate hydrogen peroxide. CROs include glyoxal oxidases (EC 1.1.3.-, GLOX) and galactose 6-oxidases (EC 1.1.3.9, GAO). GLOXes typically function on aldehydes such as methylglyoxal to produce acids. One GLOX from Phanerochaete chrysospoium primarily accepts alpha-dicarbonyl and alpha-hydroxycarbonyls. Pycnoporus cinnabainus expresses three GLOXes, one of which has been found to function on methylglyoxal while the other two of which show high catalytic efficiency for glyoxylic acid. GAOs typically function on the C6 or similar alcohols of galactose or other sugars to produce aldehydes. By far, the most characterized CRO is GAO from Fusarium graminearum. Two additional CROs capable of oxidizing aliphatic alcohols were discovered in Colletotrichum graminicola and C. gloeospoioides (CgrAlcOx and CglAlcOx). An aryl-alcohol oxidase (CgrAAO) was also discovered in C. graminicola.

CROs have been categorized as ‘green’ small-molecule oxidation catalysts as they lack dependence on an organic cofactor and require only molecular oxygen as a cosubstrate. In addition, the capacity of these enzymes to generate hydrogen peroxide may extend their applicability to catalysts for the degradation of lignocellulosic material.

In one or more aspects, the CRO is a galactose oxidase (GAO). GAO has an active site typically composed of a single copper atom coordinated to an axial tyrosine, two histidines, and an unusual cross-linked cysteine-tyrosine unit that can be oxidized to form a stable radical. A tryptophan stacked over the tyrosyl-cysteine is thought to account for further stabilization of the free radical. The catalytic cycle for GAO is split into two half-reactions where a single electron is transferred in each half.

In one or more aspects, glucose is contacted with a CRO under conditions suitable for the formation of an oxidized product. For example, conditions suitable for the formation of an oxidized product may include an amount of glucose of from about 0.1 weight per volume percent (w/v %) to about 60 w/v %, alternatively from about 5 w/v % to about 50 w/v % or alternatively from about 10 w/v % to about 40 w/v % and an amount of catalyst (e.g., CRO) of from about 0.1 milligrams/liter (mg/L) to about 30,000 mg/L, alternatively from about 5 mg/L to about 500 mg/L, or alternatively from about 10 mg/L to about 100 mg/L based on desired throughput of the reaction. Further reaction conditions may include a temperature ranging from about 1° C. to about 70° C., alternatively from about 5 to about 30° C. or alternatively from about 10° C. to about 25° C. in an aqueous media such as a phosphate buffer at a pH of from about 5° C. to about 10° C., alternatively from about 6 to about 9 or alternatively from about 7 to about 8.5. In one or more aspects, the oxidation of glucose is carried out in the presence of an oxidizing agent such as oxygen. In one or more aspects, the CRO is defined by any of SEQ ID No. 1 through SEQ ID NO. 3. In one or more aspects, the oxidized product comprises glucodialdose (GDA). In some aspects, GDA is used in subsequent reactions without further processing.

With reference to FIG. 1, in one or more aspects, GDA is condensed with a nitrogen-containing compound under conditions suitable for the formation of glucodioxime. Nonlimiting examples of nitrogen-containing compounds suitable for use in the present disclosure include hydroxylamine, hydroxylamine hydrochloride, hydroxylamine sulfate, alkyl hydroxylamines, ammonia, or combinations thereof. Conditions suitable for the condensation of GDA with a nitrogen-containing compound include an amount of nitrogen-containing compound of from about 2 equivalents (eq.) to about 30 eq., alternatively from about 2 eq. to about 10 eq. or alternatively from about 2.1 eq. to about 2.5 eq. and an amount of GDA of from about 1 weight percent (wt. %) to about 50 wt. %, alternatively from about 5 wt. % to about 30 wt. %, or alternatively from about 10 wt. % to about 20 wt. % based on the total weight of the concentration from the enzymatic reaction and further processing. Reaction conditions suitable for the formation of glucodioxime may include a temperature ranging from about 0° C. to about 100° C., alternatively from about 50° C. to about 100° C., or alternatively from about 80° C. to about 100° C. in an aqueous media such as water or water/ethanol mixtures at a pH of from about 4 to about 12, alternatively from about 7 to about 11, or alternatively from about 8 to about 8.5.

With reference to FIG. 1, glucodioxime may be reduced in the presence of hydrogen to form glucodiamine. In some aspects, the reduction of glucodioxime in the presence of hydrogen is catalyzed by a hydrogenation catalyst. In one or more aspects, a hydrogenation catalyst suitable for use in the present disclosure comprises a transition metal. In such aspects, the hydrogenation catalyst may further be characterized as heterogeneous. Hereinafter, the hydrogenation catalyst is termed the H-cat.

In one or more aspects, a H-cat for use in the present disclosure comprises (i) a transition-metal compound or transition-metal salt and (ii) a support material. For example, the H-cat may comprise Fe, Cu, Rh, Re, Ir, Co, Ni, Pt, Pd, Au, Ru, oxides thereof, and combinations thereof. In one or more aspects, the support material comprises an inert or substantially inert material such as glass, titania, silica, alumina, ceramic, or carbon. The metal loadings can range from about 0.1 weight percent (wt. %) to about 90 wt. %, or about 0.15 wt. % to about 80 wt. % based on the total weight of the catalyst. In some aspects, the H-cat is a bi-metallic catalyst. Nonlimiting examples of bimetallic catalysts suitable for use in the present disclosure include Au/Rh, Au/Ir, Au/Pd, and Au/Pt. In some aspects, the H-cat is a supported bimetallic catalyst wherein the support are of the type previously disclosed herein. In such aspects, the metal content of the H-cat may be equal to or less than about 80 wt. % where the percentage of each metal used for bimetallic catalysts varies between about 0.1 wt. % and about 100 wt. %.

In an alternative aspect, glucodioxime is used to form glucodinitrile which is subsequently hydrogenated to form glucodiamine. This is depicted schematically in FIG. 2. With reference to FIG. 2, glucodioxime is supplied as a reactant for the production of glucodiamine. For example, glucodioxime may be the product of a condensation reaction between an aldehyde and a hydroxylamine as disclosed herein. Alternatively, glucodioxime, from any suitable source, is provided as a reactant. In one or more aspects, glucodioxime is contacted with a dehydratase under conditions suitable for double dehydration of the molecule to form glucoditnitrile. In such aspects, the dehydratase may comprise one or more aliphatic aldoxime dehydratases (EC 4.8.1.2) which are enzymes that catalyze the dehydration of aliphatic aldoximes [R—CH═N—OH] to produce the corresponding aliphatic nitrile[R—C≡N] and H₂O. The systematic name of this enzyme class is aliphatic aldoxime hydro-lyase (aliphatic-nitrile-forming), abbreviated OXD* where * is a letter designating the subtype of dehydratase.

All OXDs contain (i) a heme B group, the iron of which must be in the form Fe(II) for activity, and (ii) a catalytic triad, typically arginine-histidine-serine. A large hydrophobic cavity in the active site allows for mutagenesis of the enzyme to accept a variety of substrates. OXDs capable of converting large aldoximes with six carbon main chains, aromatic rings, or heteroatoms are attractive targets as starting points for engineering activity to accept a glucodioxime substrate to form a dinitrile. In an aspect, the OXD* is OxdB sourced from Bacillus sp. 0xB-1, OxdRG from Rhodococcus globerulus A-4, OxdFG from Fusarium graminearum MAFF305135, OxdRE from Rhodococcus erythropolis (previously sp. N-771), OxdA from P. chloroaphis B23, OxdBr1 from Bradyrhizobium sp. LTSPM299, and OxdK from Pseudomonas sp K-9. In one or more aspects, the OXD is defined by any of SEQ ID No. 4 through SEQ ID NO. 11.

In an aspect, an OXD* suitable for use in the present disclosure has been engineered to accept glucodioxime as a substrate. In an aspect, glucodioxime is converted to glucodinitrile facilitated by an engineered OXD*. In one or more aspects, glucodioxime is contacted with an OXD* under conditions suitable for the formation of glucodinitrile. Conditions suitable for the formation of glucodinitrile include an amount of glucodioxime of from about 0.01 w/v % to about 50 w/v %, alternatively from about 1 w/v % to about 40 w/v % or alternatively from about 5 w/v % to about 30 w/v % and an amount of OXD* of from about 0.1 mg/L to about 30,000 mg/L, alternatively from about 5 mg/L to about 500 mg/L or alternatively from about 10 mg/L to about 100 mg/L based on desired throughput of the reaction. Reaction conditions suitable for the formation glucodinitrile may include a temperature ranging from about 1° C. to about 100° C., alternatively from about 10° C. to about 70° C. or alternatively from about 20° C. to about 50° C. in an aqueous media such as a phosphate buffer with 10 percent volume/volume solvent (e.g., dimethyl sulfoxide or acetone and 5 mM Na₂S₂O₄) at a pH of from about 4 to about 10, alternatively from about 5 to about 9 or alternatively from about 6 to about 8.

With reference to FIG. 2, glucodinitrile may be hydrogenated in the presence of a hydrogenation catalyst and hydrogen to form glucodiamine. In such aspects, the hydrogenation catalyst is of the type previously disclosed herein. Conditions suitable for the hydrogenation of glucodinitrile in the presence of hydrogen include a temperature of from about 50° C. to about 250° C., alternatively from about 100° C. to about 200° C. or alternatively from about 120° C. to about 150° C., a reaction medium comprising a suitable solvent (e.g., water, ethanol, or water/ethanol mixtures, or other organic solvents) where the glucodinitrile is present in an amount of from about 1 wt % to about 50 wt %, alternatively from about 5 wt % to about 30 wt % or alternatively from about 10 wt % to about 20 wt % based on the total weight of the final products. The product, glucodiamine, may be further processed as needed to meet some user and/or application goals. For example, the glucodiamine may be subjected to one or more purification techniques and concentration.

In one or more aspects, in reactions catalyzed by a CRO, a small molecule activator (SMA) and a single electron oxidizer (SEO) may be present along with the CRO, both of which may facilitate the catalytic activity of the CRO. Specifically, the CRO active site typically comprises a single copper atom coordinated to an axial tyrosine, two histidines, and an unusual cross-linked cysteine-tyrosine unit that can be oxidized to form a stable radical. Not intending to be bound by theory, a tryptophan stacked over the tyrosyl-cysteine is thought to account for further stabilization of the free radical. The catalytic cycle for a CRO is split into two half-reactions. Occasionally, a single electron is transferred to the cysteine-tyrosine radical resulting in an inactive, “semi-reduced” CRO. In one or more aspects, the SEO comprises a peroxidase such as horseradish peroxidase, or ferricyanide. In one or more aspects, the SMA is a molecule that (i) is capable of stabilizing a free radical, (ii) can serve as a substrate for the SEO in a single electron oxidation reaction, and iii) is capable of restoring the active state of a CRO as evidenced by detectable activity (formation of product or oxygen consumption). Nonlimiting examples of SMAs suitable for use in the present disclosure include 1,2-benzisothiazol-3(2H)-one (BIT), L-tryptophan, 2-mercaptobenzothiazole, L-histidine, methylchloroisothiazolinone, o-dianisidine, 2,2′-azino-bis(3-ethylbenzothiazoline-6-sulfonic acid (ABTS), 4-aminoantipyrine, L-tyrosine, (2,2,6,6-tetramethylpiperidin-1-yl)oxyl, chloromethylisothiazolinone, 4-thiazolecarboxylic acid, Sunset yellow FCF, tartrazine, p-benzoquinone, dicoumarol, phthalimide, saccharin, phthalic anhydride, erythrosine B, 2-aminobenzothiazole, thiabendazole, 2-hydroxybenzothiazole, phenothiazine, 6-aminobenzothiazole, indigo carmine, naphthalimide, 2-aminothiazole, thiazole, 2H-1,4-benzothiazin-3(4H)-one, 2-oxindole, beta-lapachone, menaquinone, thiamine, 4-methyl-5-thiazoleethanol, Allura Red AC, menadione, p-cresol, Fast green FCF, Brilliant Blue FCF, methylisothiazolinone, caffeine, veratryl alcohol, and combinations thereof.

In some aspects, enzymes suitable for use in the present disclosure may further include one or more purified cofactors. Herein a cofactor refers to non-protein chemical compound that modulates the biological activity of the biocatalyst. Many enzymes require cofactors to function properly. Nonlimiting examples of purified enzyme cofactors suitable for use in the present disclosure include thiamine pyrophosphate, NAD+, NADP+, pyridoxal phosphate, methyl cobalamin, cobalamine, biotin, Coenzyme A, tetrahydrofolic acid, menaquinone, ascorbic acid, flavin mononucleotide, flavin adenine dinucleotide, and Coenzyme F420. Such cofactors may be included in the enzyme preparation and/or be added at various points during the reaction. In some aspects, cofactors included with the enzyme preparation may be readily regenerated with oxygen and/or may remain stable throughout the lifetime of the enzyme(s).

As will be understood by one of ordinary skill in the art with the benefit of the present disclosure, reactions of the type disclosed herein may result in the production of byproducts (e.g., hydrogen peroxide) that can detrimentally impact other components of the reaction mixture. For example, hydrogen peroxide may degrade the enzyme resulting in a loss of catalytic activity. In such aspects, mitigation of the detrimental effects of hydrogen peroxide may be carried out such as by the introduction of a catalase (E.C. 1.11.1.61), the use of a hydrogen peroxide-resistant enzyme or combinations thereof.

In some aspects, an enzyme of the type disclosed herein is a wild type enzyme, a functional fragment thereof, or a functional variant thereof. “Fragment” as used herein is meant to include any amino acid sequence shorter than the full-length enzyme, but where the fragment maintains a catalytic activity sufficient to meet some user or process goal. Fragments may include a single contiguous sequence identical to a portion of the biocatalyst sequence. Alternatively, the fragment may have or include several different shorter segments where each segment is identical in amino acid sequence to a different portion of the amino acid sequence of the enzyme but linked via amino acids differing in sequence from the enzyme. Herein, a “functional variant” of the enzyme refers to a polypeptide which has at one or more positions of an amino acid insertion, deletion, or substitution, either conservative or non-conservative, and wherein each of these types of changes may occur alone, or in combination with one or more of the others, and/or one or more times in a given sequence but retains catalytic activity.

In the alternative or in combination with the aforementioned mutations, the enzyme may be mutated to improve the catalytic activity. Mutations may be carried out to enhance the protein or a homolog activity, increase the protein stability in the presence of substrates and products (e.g., hydrogen peroxide) and increase protein yield.

Herein, reference has been made to “sources” of enzyme. It is to be understood this refers to the biomolecule as expressed by the named organism. It is contemplated the enzyme may be obtained from the organism or a version of said enzyme (wildtype or recombinant) and provided as a suitable construct to an appropriate expression system.

In an aspect, an enzyme of the type disclosed herein may be cloned into an appropriate expression vector and used to transform cells of an expression system such as E. coli, Saccharomyces sp., Pichia sp., Aspergillus sp., Trichoderma sp., or Myceliophthora sp. A “vector” is a replicon, such as plasmid, phage, viral construct or cosmid, to which another DNA segment may be attached. Vectors are used to transduce and express a DNA segment in cells. As used herein, the terms “vector” and “construct” may include replicons such as plasmids, phage, viral constructs, cosmids, Bacterial Artificial Chromosomes (BACs), Yeast Artificial Chromosomes (YACs) Human Artificial Chromosomes (HACs) and the like into which one or more gene expression cassettes may be or are ligated. Herein, a cell has been “transformed” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell.

In an aspect, the gene of an enzyme disclosed herein is provided as a recombinant sequence in a vector where the sequence is operatively linked to one or more control or regulatory sequences. “Operatively linked” expression control sequences refer to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and are used herein refer to polynucleotide sequences which affect the expression of coding sequences to which they are operatively linked. Expression control sequences are sequences that control the transcription, post-transcriptional events, and translation of nucleic acid sequences. Expression control sequences include appropriate transcription initiation, termination, promoter, and enhancer sequences; efficient RNA processing signals such as splicing and polyadenylation signals; sequences that stabilize cytoplasmic mRNA; sequences that enhance translation efficiency (e.g., ribosome binding sites): sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

In one or more aspects, glucodiamine prepared as described herein is used as a platform molecule in the preparation of a polyamide polymer (e.g., Nylon). In one or more aspects, a composition is provided for a biodegradable polyamide using hexamethylenediamine (also known as hexamethylenediamineor HDMA), adipic acid, and glucodiamine. The glucodiamine may be introduced in strategic points in the polyamide production process to enable the generation of polymers characterized by a higher biodegradability and recyclability. Glucodiamine can be introduced to the polymer production process in any suitable manner such as in a structural fashion or in a randomized fashion depending on some user and/or process desired objective. For example, glucodiamine may be introduced to the polymer production process to generate a polyamide for a specific application or to achieve some desired level of polymer biodegradability.

In one or more aspects, glucodiamine may function as a chemical capture solvent for carbon dioxide sequestration. CO₂ sequestration is the process of capturing, securing and storing carbon dioxide. For example, post-combustion CO₂ may be contacted with a glucodiamine solution to form a CO₂-glucodiamine adduct. The CO₂-glucodiamine adduct may be safely stored in any suitable location, for example an underground geological formation. In an alternative aspect, the CO₂ when contacted with a glucodiamine solution is subjected to conditions such that the CO₂ reacts with a primary amine to form a carbamate, which can also be stored in any suitable location.

The methods and compositions disclosed herein provide novel pathways for the synthesis of new polymeric materials that remain unexplored. The resulting polymers would be green when compared to petroleum-based polymers as they are the product of reactants obtained from renewable resources (e.g., sugars from biomass).

Additional nonlimiting aspects of the present disclosure are as follows described hereinbelow.

A first aspect which is a method of preparing glucodiamine, comprising contacting glucose with an enzyme oxidation catalyst under conditions suitable for the formation of glucodialdose; contacting glucodialdose with a nitrogen-containing compound under conditions suitable for the formation of glucodioxime; and reducing glucodioxime under conditions suitable for the formation of glucodiamine.

A second aspect which is the method of the first aspect wherein the enzyme oxidation catalyst comprises a copper radical oxidase.

A third aspect which is the method of the second aspect, wherein the copper radical oxidase comprises galactose oxidase, mutants thereof or combinations thereof.

A fourth aspect which is the method of any of the first through third aspects, wherein glucose is derived from a biomass.

A fifth aspect which is the method of any of the first through fourth aspects wherein conditions suitable for the formation of glucodialdose comprise a temperature of from about 1° C. to about 70° C. and a pH of from about 5 to about 10.

A sixth aspect which is the method of any of the first through fifth aspects wherein conditions suitable for the formation of glucodialdose further comprise the presence of a catalase.

A seventh aspect which is the method of any of the first through sixth aspects wherein conditions suitable for the formation of glucodialdose further compnse a small molecule activator, a single electron oxidizer or combinations thereof.

An eighth aspect which is the method of any of the first through seventh aspects wherein the nitrogen-containing compound comprises hydroxylamine, hydroxylamine hydrochloride, hydroxylamine sulfate, alkyl hydroxylamines, ammonia, or combinations thereof.

A ninth aspect which is the method of any of the first through eighth aspect wherein conditions suitable for the formation of glucodioxime comprise a temperature of from about 0° C. to about 100° C. and a pH of from about 4 to about 12.

A tenth aspect which is the method of any of the first through ninth aspects wherein conditions suitable for the formation of glucodiamine comprise hydrogen and a hydrogenation catalyst.

An eleventh aspect which is the method of the tenth aspect wherein the hydrogenation catalyst comprises at least one transition metal and a catalyst support.

A twelfth aspect which is the method of any of the ninth through tenth aspects wherein conditions suitable for the formation of glucodiamine comprise a temperature of from about 50° C. to about 250° C.

A thirteenth aspect which is a method of preparing glucodiamine, comprising: contacting glucodioxime with a dehydration catalyst under conditions suitable for the formation of glucodinitrile; and reducing glucodinitrile under conditions suitable for the formation of glucodiamine.

A fourteenth aspect which is the method of the thirteenth aspect wherein the dehydration catalyst comprises aliphatic aldoxime dehydratase.

A fifteenth aspect which is the method of the fourteenth aspect wherein the aliphatic aldoxime dehydratase is mutated.

A sixteenth aspect which is the method of any of the thirteenth through fifteenth aspects, wherein conditions suitable for the formation of glucodiamine comprise hydrogen and a hydrogenation catalyst.

A seventeenth aspect which is the method of any of the thirteenth through sixteenth aspects wherein the hydrogenation catalyst comprises at least one transition metal and a catalyst support.

An eighteenth aspect which is the method of any of the thirteenth through seventeenth aspects wherein the hydrogenation catalyst is a supported bimetallic catalyst.

A nineteenth aspect which is a chemoenzymatic method of producing a bio-based amide platform chemical, comprising contacting glucose with a biocatalyst under conditions suitable for the formation of glucodialdose; contacting glucodialdose with a base under conditions suitable for the formation of glucodioxime; and contacting glucodioxime with a hydrogenation catalyst in the presence of hydrogen under conditions suitable for formation of glucodiamine.

A twentieth aspect which is the method of the nineteenth aspect wherein the biocatalyst comprises a mutated galactose oxidase.

A twenty-first aspect which is the method of any of the nineteenth through twentieth aspects wherein the hydrogenation catalyst comprises a metal.

### EXAMPLES

The disclosure having been generally described, the following examples are given as particular aspects of the disclosure and to demonstrate the practice and advantages thereof. It is understood that the examples are given by way of illustration and are not intended to limit the specification of the claims to follow in any manner.

### Example 1

Generation of GAO Mutant for Producing Glucodialdose from Glucose

A GAO mutant was engineered that is capable of converting glucose to glucodialdose. Following directed evolution and rational enzyme engineering, the improved GAO mutant exhibits a specific activity of greater than 35 U mg⁻¹ on glucose.

### Directed Evolution

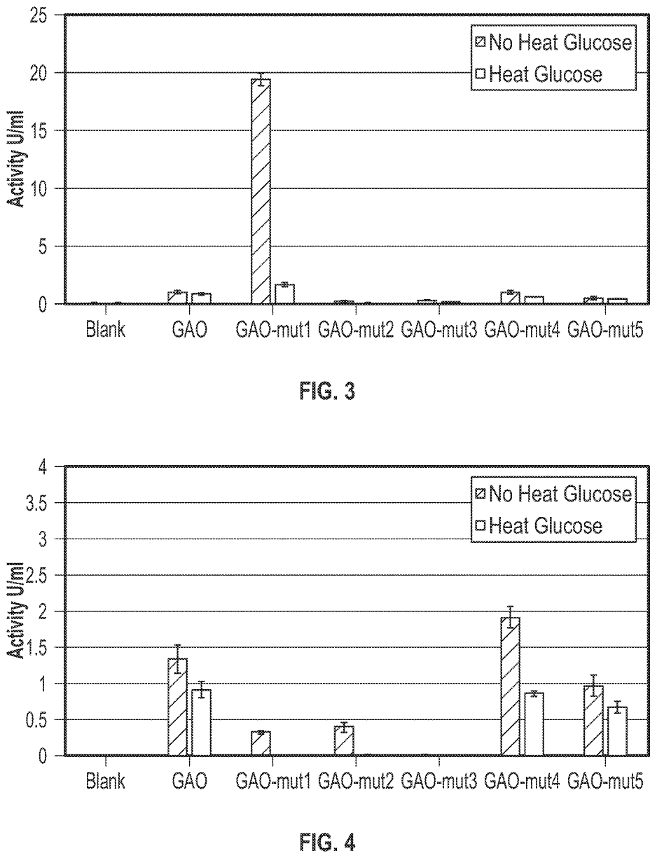

Directed evolution of thirty sites within 10 Å of the catalytic copper was performed on a parent sequence reported containing the following added mutations: 1) R330, Q406T, W290F to introduce less than 1 U mg⁻¹ activity on glucose to GAO, 2) C383S discovered to lower the KM of the enzyme on galactose, and 3) Y405F and Q406E to enhance activity on a D-N-acetyl glucosamine substrate. Other mutations described in Table I were found to have neutral or deleterious effects on glucodialdose-generating activity. We named the new combination sequence GAO-Mut1. A graph of the specific activity of previously described GAO mutants on glucose is presented in FIG. 3. The full sequence of the expressed construct is given as follows:

>GAO-Mut1
MGHHHHHHSSGHIEGRHMASAPIGSAIPRNNWAVTCDSAQSGNECN
KAIDGNKDTFWHTFYGANGDPKPPHTYTIDMKTTQNVNGLSVLPR
QDGNQNGWIGRHEVYLSSDGTNWGSPVASGSWFADSTTKYSNFET
RPARYVRLVAITEANGQPWTSIAEINVFQASSYTAPQPGLGRWGP
TIDLPIVPÅAAAIEPTSGRVLMWSSYRNDAFEGSPGGITLTSSWD
PSTGIVSDRTVTVTKHDMFCPGISMDGNGQIVVTGGNDAKKTSLY
DSSSDSWIPGPDMQVARGYQSSATMSDGRVFTIGGSFSGGVFEKN
GEVYSPSSKTWTSLPNAKVNPMLTADKQGLYKSDNHAWLFGWKKG
SVFQAGPSTAMNWYYTSGSGDVKSAGKRQSNRGVAPDAMSGNAVM
YDAVKGKILTFGGSPDFEDSDATTNAHIITLGEPGTSPNTVFASN
GLYFARTFHTSVVLPDGSTFITGGQRRGIPFEDSTPVFTPEIYVP
EQDTFYKQNPNSIVRAYHSISLLLPDGRVFNGGGGLCGDCTTNHF
DAQIFTPNYLYDSNGNLATRPKITRTSTQSVKVGGRITISTDSSI
SKASLIRYGTATHTVNTDQRRIPLTLTNNGGNSYSFQVPSDSGVA
LPGYWMLFVMNSAGVPSVASTIRVTQ

TABLE I

| Name | Staring Mut | Addition mutations |
|---|---|---|
| M1 | wild-type | S10P M70V P136P G195E V494A |
| | | N535D |
| M-RQW | M1 | R330K Q406T W290F I463P |
| GAO | M-RQW | C383S |
| GAO-mut1 | GAO | Y405F Q406E |
| GAO-mut2 | GAO | F194T |
| GAO-mut3 | GAO | C383E |
| GAO-mut4 | GAO | N245R |
| GAO-mut5 | GAO | Q326E Y329K |

### Example 2

Selected positions in GAO-Mut1 were mutated via the QUIKCHANGE method to all 20 amino acids using primers containing NNS codons. The constructs were then screened in the following manner colonies were picked and used to inoculate one well each in a 96-well deepwell plate charged with LB. The grown clones were then used to inoculate autoinduction media in a separate 96-well deepwell plate for protein expression. Harvested cells were lysed with Bacterial Protein Extraction Reagent (B-PER) and the lysate was then screened for oxidase activity using a colorimetric ABTS assay which detects hydrogen peroxide. In short, lysate was assayed for activity with and without exposure to heat. To assay activity in the absence of a heat challenge, lysate was diluted 50 times. A volume of 5 μL of the diluted lysate was combined with ABTS assay solution (final concentration of 2% w/v glucose, 0.0125 mg/ml horseradish peroxidase, 50 mM sodium phosphate buffer at pH 8, 0.05% ABTS) to a final volume of 200 μL and the change in absorbance at 405 nm was monitored until the reaction was complete. To assay residual activity after a heat challenge, 50 μL lysate was incubated for ten minutes at 50° C. and 20 μL of the heat-treated lysate was added to the ABTS solution before monitoring change in absorbance at 405 nm. Specific activity was calculated from the formulas below using the linear portion of the curve to measure ΔA405/min and taking the extinction coefficient of ABTS at 405 nm as 36.8 mM⁻¹·cm⁻¹.

$$Units {mg}^{- 1} = \frac{Δ {A}_{405} {min}^{- 1}}{36.8 \times ( path length in cm ) \times ( mg enzyme ) / ( ml reaction mixture )}$$

$$Units {ml}^{- 1} = \frac{{Δ Α}_{405} {min}^{- 1}}{36.8 \times ( path length in cm ) \times ( ml enzyme ) / ( ml reaction mixture )}$$

Mutant lysates exhibiting a ΔA405/min greater than GAO-Mut1 were chosen for further characterization. Following identification of the mutation by DNA sequencing, hits were expressed, purified, and assayed for specific activity and thermostability as assessed by the temperature at which one half maximal activity was observed (T₅₀). Mutants were purified by from a 5 ml culture with auto-induction medium in 25 well plate. Harvested cells were lysed with Bacterial Protein Extraction Reagent (B-PER) and the lysate was span down with 15,000 rcf for 30 min at 4° C. The lysate supernatant was used to protein purification with HisPur™ Ni-NTA Spin Plates. The eluted protein sample was diluted with 100 mM potassium phosphate buffer pH=7.5 with 0.5 mM CuSO₄, and specific activity was measured using the ABTS assay outlined above. T₅₀ was measured by heating protein in the absence of substrate, cooling, and then measuring residual activity using the ABTS assay. Heating was accomplished by diluting the protein to a concentration of 2.5 mg/L in a volume of 100 mM phosphate buffer at pH 7.5, aliquoting 50 μL into a row of a 96-well PCR plate, and incubating over a temperature gradient sufficient to capture maximal and minimal enzyme performance for ten minutes. Promptly after heating, the mixture was cooled on ice and the ΔA405/min of 20 μL of enzyme solution in 200 μL final volume of ABTS solution was measured as described above.

Hits were purified, tested for activity and T₅₀, and recombined to generate a final best mutant from the directed evolution step. Promising point mutants that could beneficially be combined in the Mut1 background included A193R, D404H, F4414Y, and A72V (Table II). These mutations were combined into a single combination mutant named GAO-Mut47 which exhibited a specific activity of 19.68 U mg⁻¹ and a T₅₀ of 56.8° C.

TABLE II

| | | | T50 | Kcat | Km |
|---|---|---|---|---|---|
| Name | Mutations | U/mg | ° C. | S − 1 | mM |
| M-RQW-S | | 1.1 | 56.8 | 31.4 | 2168.3 |
| GAO-mut1 | Y405F Q406E | 14.0 | 51.8 | 30.2 | 93.1 |
| GAO-mut6 | Y405F Q406E S383C | 6.1 | 41.5 | 36.6 | 412.0 |
| GAO-mut7 | Y405F Q406E F441Y | 16.9 | 53.6 | 27.3 | 42.7 |
| GAO-mut8 | Y405F Q406E D404H | 15.0 | 53.7 | 30.7 | 83.9 |
| GAO-mut9 | Y405F Q406E G461A | 13.4 | 53.2 | 27.8 | 83.7 |
| GAO- | Y405F Q406E I462R | 12.1 | 53.2 | 31.6 | 130.7 |
| mut10 | | | | | |
| GAO- | Y405F Q406E A172V | 21.2 | 48.5 | 39.5 | 72.6 |
| mut11 | | | | | |
| GAO- | Y405F Q406E A193R | 15.4 | 56.3 | 28.0 | 64.8 |
| mut12 | | | | | |
| GAO- | Y405F Q406E A193T | 14.6 | 53.8 | 30.4 | 75.5 |
| mut13 | | | | | |
| GAO- | Y405F Q406E D404H F441Y | 18.8 | 55.0 | 26.5 | 29.7 |
| mut14 | | | | | |
| GAO- | Y405F Q406E G461A I462R | 12.2 | 54.0 | 24.8 | 79.1 |
| mut15 | | | | | |
| GAO- | Y405F Q406E D404H F441Y G461A | 18.2 | 55.3 | 23.6 | 25.5 |
| mut17 | I462R | | | | |
| GAO- | Y405F Q406E A193T D404H F441Y | 18.1 | 56.6 | 24.1 | 28.0 |
| mut18 | G461A I462R | | | | |
| GAO- | Y405F Q406E A193T D404H F441Y | 13.3 | 46.3 | 24.5 | 70.8 |
| mut19 | G461A I462R S383C | | | | |
| GAO- | Y405F Q406E A193T D404H F441Y | 21.4 | 37.9 | 35.6 | 58.2 |
| mut20 | G461A I462R S383C A172V | | | | |
| GAO- | Y405F Q406E F441Y G461A I462R | 18.3 | 53.8 | 24.2 | 29.6 |
| mut21 | | | | | |
| GAO- | Y405F Q406E A193T D404H F441Y | 23.6 | 51.5 | 29.5 | 26.4 |
| mut22 | G461A I462R A172V | | | | |
| GAO- | Y405F Q406E A193R D404H F441Y | 21.1 | 57.5 | 27.2 | 26.8 |
| mut23 | G461A I462R A172V | | | | |
| GAO- | Y405F Q406E A193R D404H F441Y | 27.3 | 56.8 | 35.0 | 25.2 |
| mut47 | A172V | | | | |
| GAO- | Y405F Q406E D404H F441Y A172V | 27.1 | 52.9 | 35.4 | 26.6 |
| mut58 | | | | | |

### Example 3

### Rational Engineering

Rational engineering of GAO to further accept a glucose substrate and identify stabilizing mutations was accomplished with a combination of computational methods based on structural and multiple sequence alignment data (MSA). Previously, we identified that GAO-M-RQW-S (the GAO-Mut1 sequence without the Y405F and Q406E mutations) could accept both glucose and gluconate as substrate. As efforts were underway to produce a GAO active on both substrates, rational design was performed on the GAO-M-RQW-S sequence rather than GAO-Mut1. Structural methods employed included applying FoldX11 (40 predicted mutations) and PROSS12 (80 mutations) to a modified form of the PDB structure 2WQ8 to contain the GAO-M-RQW-S mutations. MSA-based predictions were made using methods applied to a 185-member MSA. This MSA was generated from an initial set of 1000 sequences curated with JALVIEW to remove sequences with 98% redundancy and retain only sequences experimentally verified as carbohydrate oxidases. 30 mutations identified by Merck in designing a GAO for synthesizing an intermediate of the HIV drug ISLATRAVIR were also added to the panel. A graph of the specific activity of previously described GAO mutants on gluconate is presented in FIG. 4.

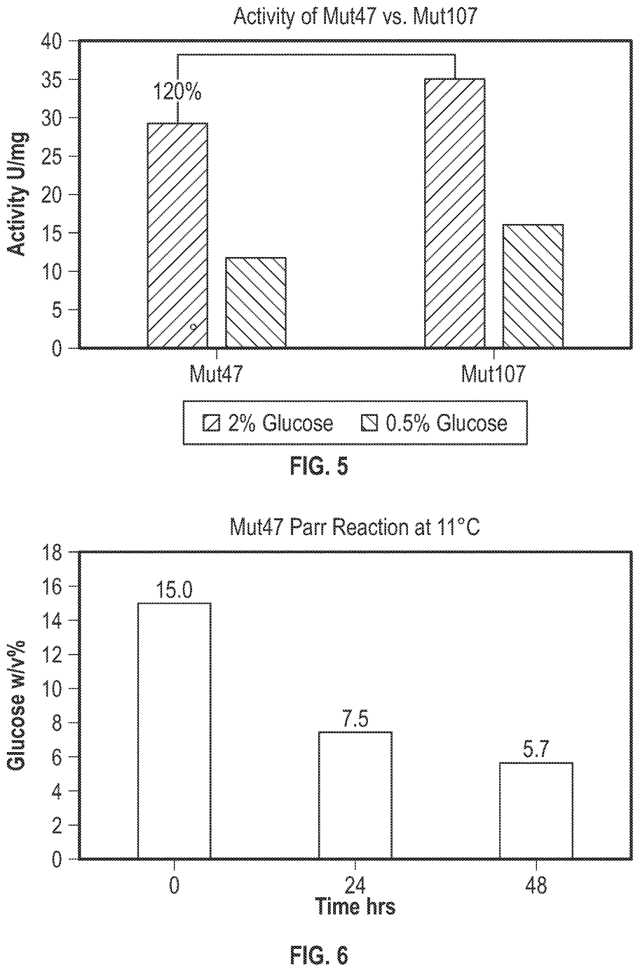

In total, 202-point mutants were screened using the same methods described above for screening the directed evolution clones. Thirty-nine hits were identified from an initial screen and sixteen were reidentified from a second round of screening. Upon generation of combo mutants in the best combination mutant from the directed evolution step (GAO-Mut47), the mutations N66S, S306A, S311F, and Q486L were identified as complementary and beneficial while N281, Y189W, S331R, A378D, and R459Q were deemed detrimental in this background (Table Ill). The final GAO-Mut107 construct containing the Mut47 mutations and N66S, S306A, S314F, and 486L exhibits a specific activity of 34.96 U mg⁻¹ on 2% glucose and a T₅₀ of 60.56° C. (FIG. 6). Additional mutations identified from machine learning algorithms were later incorporated to generate GAO-mut142 and GAO-mut164. A graph of the specific activity of GAO-Mut47 and 107 on 0.5 and 2% Glucose FIG. 5.

TABLE III

| | | New | | Fold | T50 |
|---|---|---|---|---|---|
| Clone | Mutations from Mut47 | Mutations | U/mg | Improvement | ° C. |
| Mut47 | | | 31.11 | 1.00 | 57.64 |
| GAO- | N28I | N28I | 30.84 | 0.99 | 56.76 |
| mut68 | | | | | |
| GAO- | N28I N66S | N66S | 33.68 | 1.08 | 59.00 |
| mut69 | | | | | |
| GAO- | N28I N66S Y189W | Y189W | 31.80 | 1.02 | 59.91 |
| mut70 | | | | | |
| GAO- | N28I N66S Y189W S306A | S306A | 32.66 | 1.05 | 59.48 |
| mut71 | | | | | |
| GAO- | N28I N66S Y189W S306A | S311F | 33.87 | 1.09 | 60.81 |
| mut72 | S311F | | | | |
| GAO- | N28I N66S Y189W S306A | S331R | 27.56 | 0.89 | 59.87 |
| mut73 | S311F S331R | | | | |
| GAO- | N28I N66S Y189W S306A | A378D | 25.57 | 0.82 | 58.94 |
| mut74 | S311F S331R A378D | | | | |
| GAO- | N28I N66S Y189W S306A | R459Q | 23.51 | 0.76 | 58.49 |
| mut75 | S311F S331R A378D R459Q | | | | |
| GAO- | N28I N66S Y189W S306A | V477D | 19.22 | 0.62 | 59.17 |
| mut76 | S311F S331R A378D R459Q | | | | |
| | V477D | | | | |
| GAO- | N28I N66S Y189W S306A | Q486L | 24.57 | 0.79 | 59.88 |
| mut77 | S311F S331R A378D R459Q | | | | |
| | V477D Q486L | | | | |
| GAO- | N66S S306A S311F Q486L | Removed | 34.96 | 1.20 | 60.56 |
| mut107a | | N28I, | | | |
| | | Y189W, | | | |
| | | S331R, | | | |
| | | A378D, | | | |
| | | R459Q, | | | |
| | | and V477D | | | |
| GAO- | N66S S306A S311F Q486L | H40C | 37.53 | 1.29 | 58.76 |
| mut142b | H40C | | | | |
| GAO- | N66S S306A S311F Q486L | L71C | 38.22 | 1.32 | 52.97 |
| mut164b | H40C L71C | | | | |

Bolded mutations are beneficial in a Mut47 background A193R D404H F441Y A172V
aData collected in a separate experiment from other data. Fold improvement is calculated compared to an internal Mut47 control.
bData collected in a separate experiment from other data. Fold improvement is calculated compared to an internal Mut47 control.

### Example 4

One-Step Parr Bomb Reaction with GAO-Mut47 to Produce D-Glucodialdose

A 50 ml reaction was conducted in a 200 mL vessel pressurized to 100 psi. The vessel was charged with 50 mM sodium phosphate pH 8 buffer, 50 μM CuSO₄, 15 w/v % glucose, 0.005 w/v % catalase, 0.001% horseradish peroxidase, and 0.001 w/v % engineered GAO. The reaction was stirred 500 rpm, 11° C. for 48 hours. Samples were taken at 0, 24, and 48 hours then assayed with HPLC to measure residual glucose.

Two-Step Parr Bomb Reaction with GAO-Mut47 to Produce L-Guluronic Acid

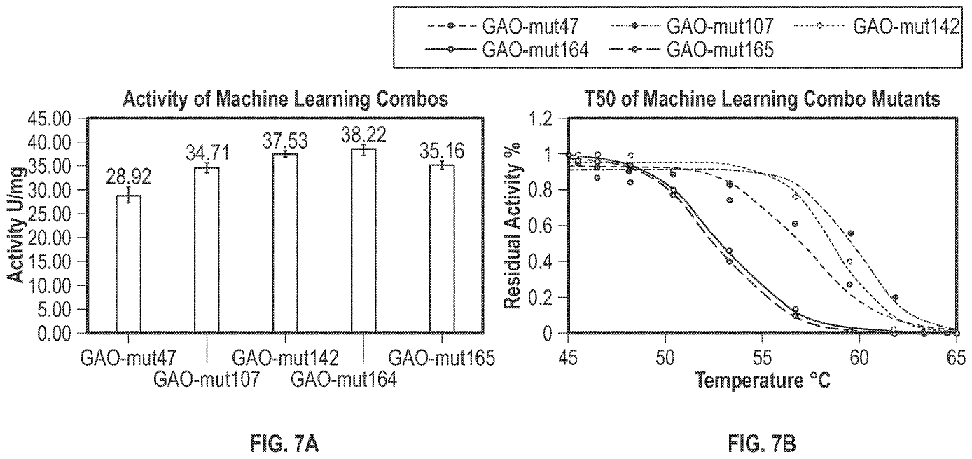

A 50 ml reaction was conducted in a 200 mL vessel pressurized to 100 psi. The vessel was charged with 50 mM sodium phosphate pH 8 buffer, 50 μM CuSO₄, 15 w/v % glucose, 0.005 w/v % catalase, 0.001% horseradish peroxidase, and 0.01 w/v % engineered GAO. The reaction was stirred 500 rpm, 11° C. for 72 hours to generate glucodialdose from glucose. In the second step, 0.002% w/v GOX and an additional 0.001% w/v catalase was added and allowed to proceed at the same conditions for another 24 hours. The reaction was periodically paused and the pH adjusted to 7. FIGS. 6 and 7 are graphs of the residual glucose concentration in the Parr reaction and the activity and stability of GAO mutants containing mutations discovered through machine learning, respectively

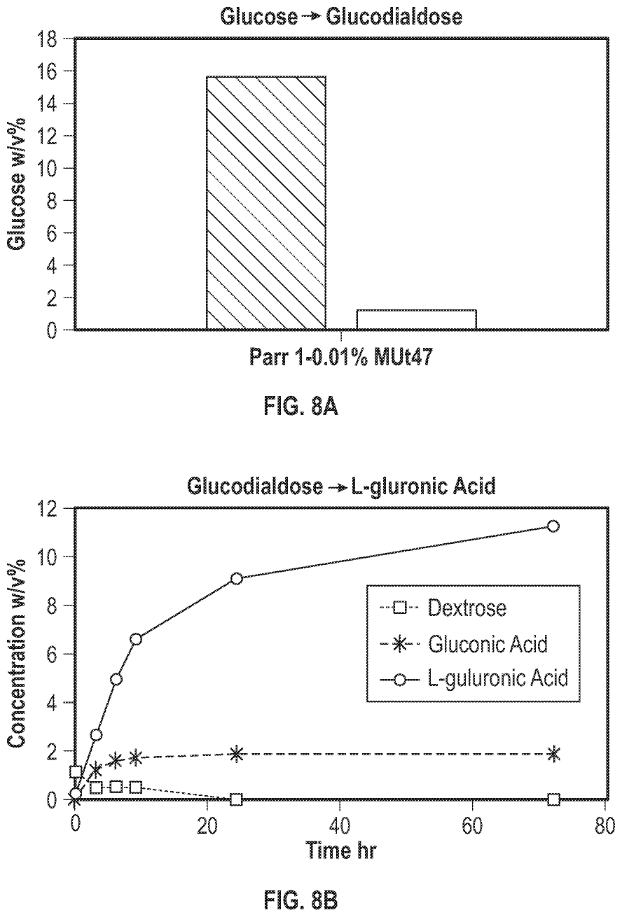

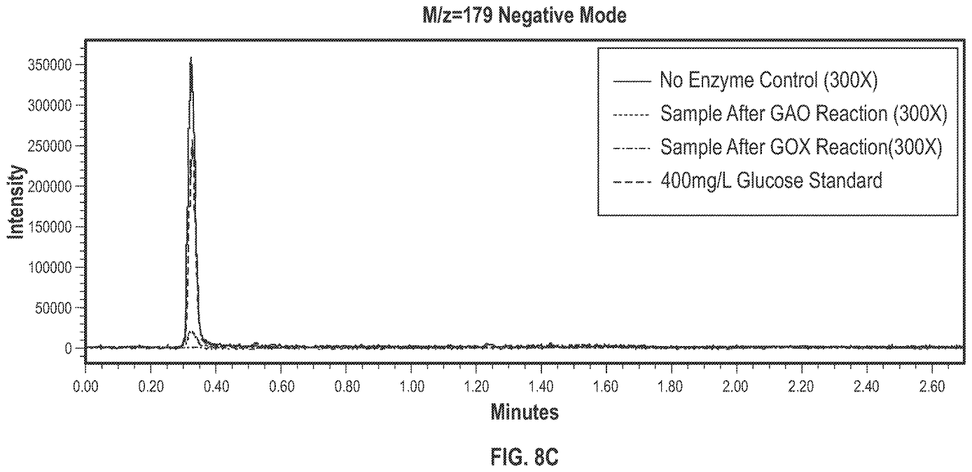

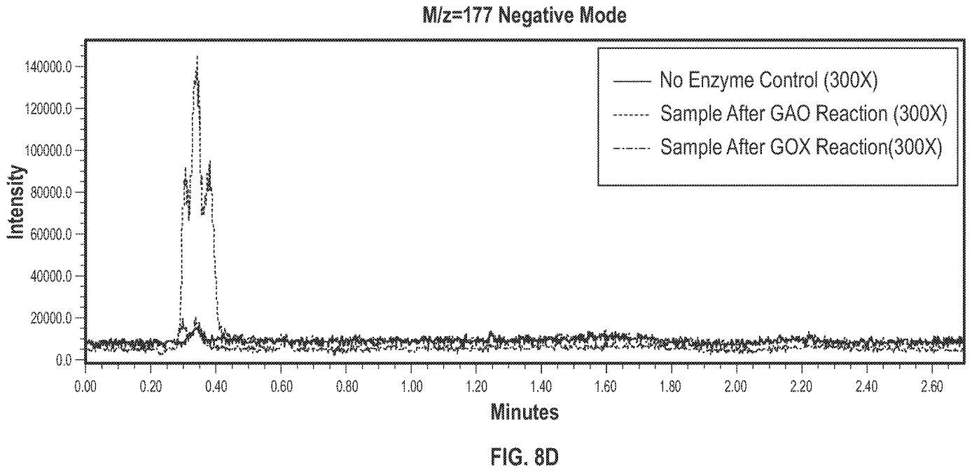

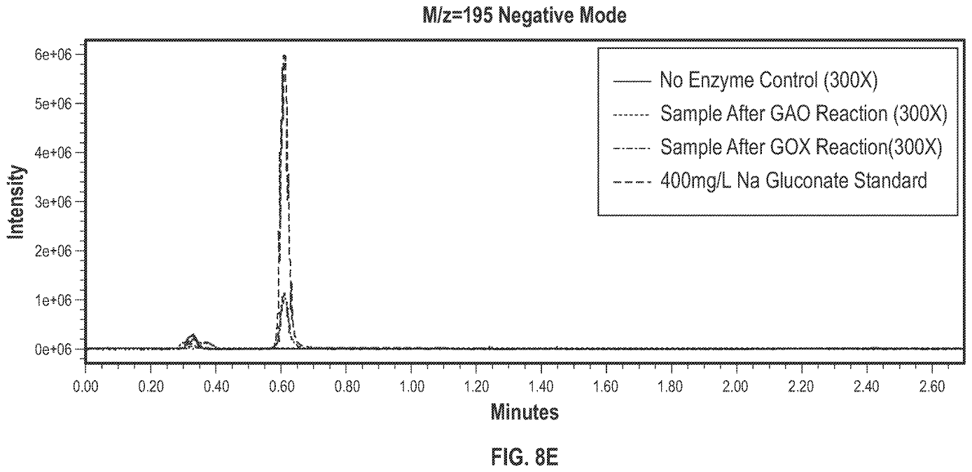

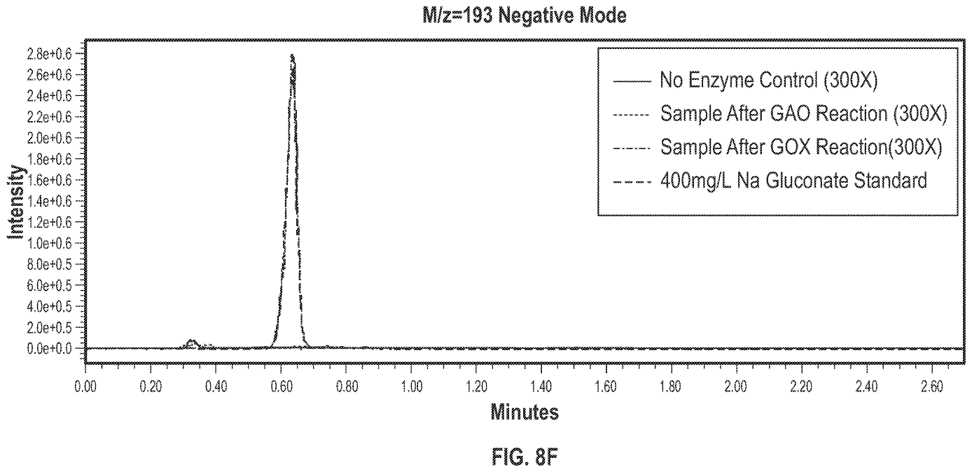

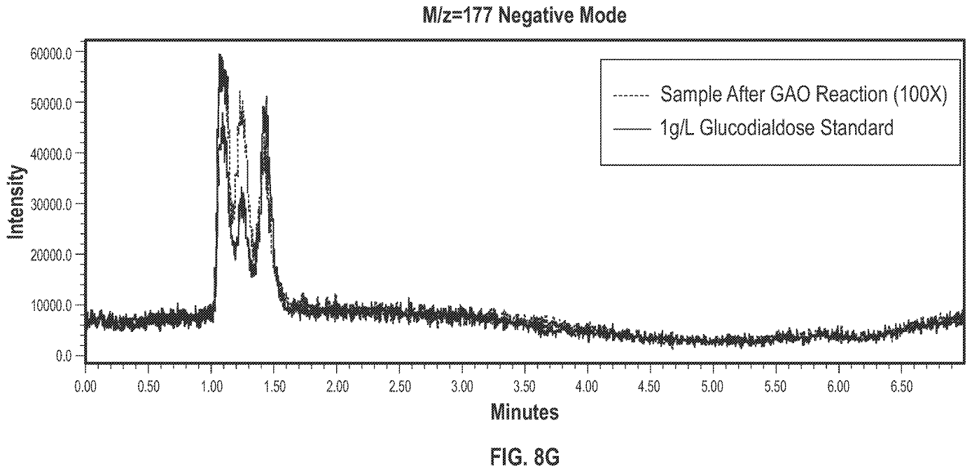

Following the reaction with GAO-Mut47, the concentration of glucose dropped from the initial loading of 16% w/v to 1.5% w/v. After addition of GOX and reaction for 24 hours, a mixture of 2.0% w/v gluconic and 12% w/v L-guluronic acid (75% molar yield). FIG. 8 depicts graphs of the HPLC and MS spectra of the indicated samples.

### Example 5

Synthesis of glucodioxime from glucodialdose was investigated. Glucodioxime was synthesized by reaction of an 8 wt % aqueous solution of glucodialdose obtained enzymatically and aqueous hydroxylamine (2.2 equivalents) at 100° C. for 20 minutes at controlled pH (typically 8.5; adjusted with HNO₃). The synthesis was optimized by ¹H-NMR integrating the characteristic peaks of the oxime that appear between 6.8 and 7.8 ppm and utilizing maleic acid as an internal standard. Nuclear magnetic resonance (NMR) quantification with maleic acid as an internal standard showed conversions of glucodialdose to glucodioxime higher than 95%. The product was isolated by lyophilization and characterized by infrared spectroscopy (IR), mass spectrometry (MS), and NMR to confirm the structure.

### Example 6

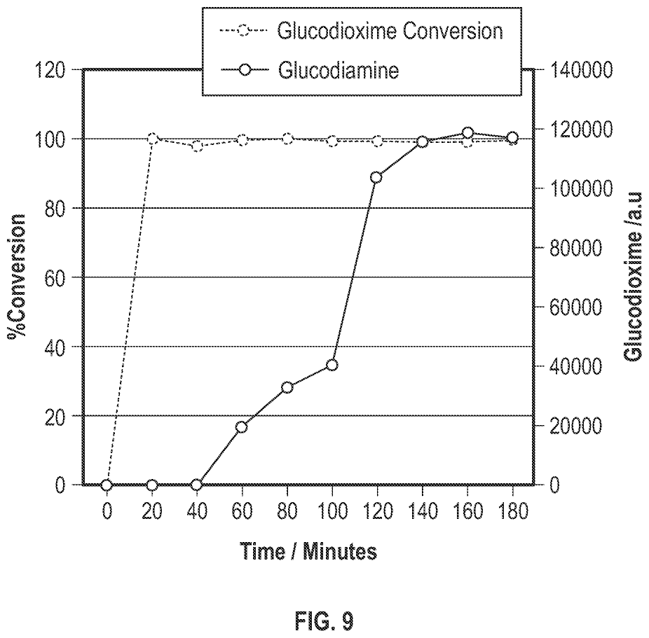

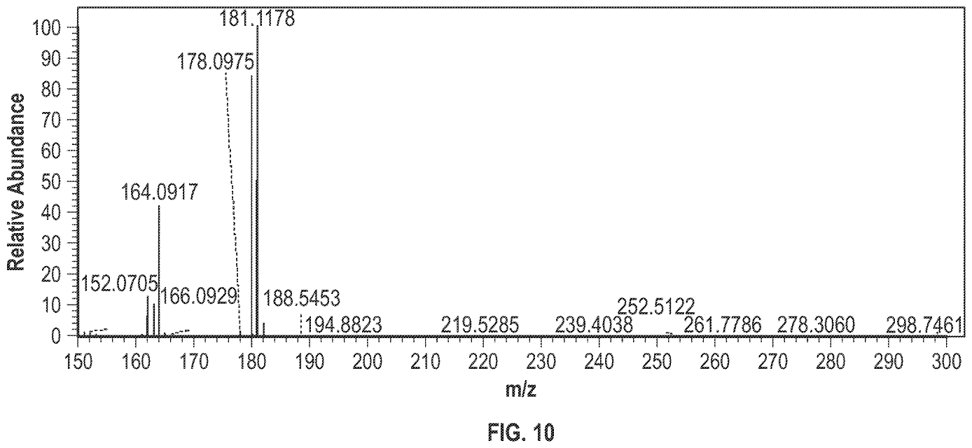

Synthesis of glucodiamine from glucodioxime was investigated. The synthesis of glucodiamine was achieved by reacting a 4 w % solution in water of glucodioxime (made following the synthesis procedures disclosed herein) in a flow reactor with a Ni catalyst in the presence of hydrogen gas. A three-phase trickle bed reactor was loaded with 100 g of a commercial Ni/NiO catalyst and pressurized at 800 psi with a gas flow rate of 0.75 standard liters per minute (SLPM). The glucodioxime solution was pumped through the reactor at a 10 mL/min liquid flow rate and heated to 150° C. The production of glucodiamine was followed by HPLC-MS and cellulose thin layer chromatography (TLC) with a solvent mixture of 8:8:1:4 pyidine/isopropanoVacetic acid/water and a 2% ninhydrin solution in ethanol stain, FIG. 9. Once the reaction reached a steady state the product was collected and analyzed. HPLC-MS data showed full conversion of the dioxime and production of the desired product along with some on-path intermediates. The final product was isolated via prep-HPLC and the fraction containing glucodiamine analyzed by HR-MS, FIG. 10, confirming that presence of the desired product. FIG. 10 is a high performance liquid chromatography-mass spectra of a product fraction containing glucodiamine that shows the exact mass of the product with an error of ~3.4 ppm.

The subject matter having been shown and described, modifications thereof can be made by one skilled in the art without departing from the spirit and teachings of the subject matter. The aspects described herein are exemplary only and are not intended to be limiting. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the present invention. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A method of preparing glucodiamine, comprising:
contacting glucose with an enzyme oxidation catalyst under conditions suitable for the formation of glucodialdose;
contacting glucodialdose with a nitrogen-containing compound under conditions suitable for the formation of glucodioxime; and
reducing glucodioxime under conditions suitable for the formation of glucodiamine.

2. The method of claim 1, wherein the enzyme oxidation catalyst comprises a copper radical oxidase.

3. The method of claim 2, wherein the copper radical oxidase comprises galactose oxidase, mutants thereof or combinations thereof.

4. The method of claim 1, wherein glucose is derived from a biomass.

5. The method of claim 1, wherein conditions suitable for the formation of glucodialdose comprise a temperature of from about 1° C. to about 70° C. and a pH of from about 5 to about 10.

6. The method of claim 1, wherein conditions suitable for the formation of glucodialdose further comprise the presence of a catalase.

7. The method of claim 1, wherein conditions suitable for the formation of glucodialdose further comprise a small molecule activator, a single electron oxidizer or combinations thereof.

8. The method of claim 1, wherein the nitrogen-containing compound comprises hydroxylamine, hydroxylamine hydrochloride, hydroxylamine sulfate, alkyl hydroxylamines, ammonia, or combinations thereof.

9. The method of claim 1, wherein conditions suitable for the formation of glucodioxime comprise a temperature of from about 0° C. to about 100° C. and a pH of from about 4 to about 12.

10. The method of claim 1, wherein conditions suitable for the formation of glucodiamine comprise hydrogen and a hydrogenation catalyst.

11. The method of claim 1, wherein the hydrogenation catalyst comprises at least one transition metal and a catalyst support.

12. The method of claim 10, wherein conditions suitable for the formation of glucodiamine comprise a temperature of from about 50° C. to about 250° C.

13. A method of preparing glucodiamine, comprising:
contacting glucodioxime with a dehydration catalyst under conditions suitable for the formation of glucodinitrile; and
reducing glucodinitrile under conditions suitable for the formation of glucodiamine.

14. The method of claim 13, wherein the dehydration catalyst comprises aliphatic aldoxime dehydratase.

15. The method of claim 13, wherein the aliphatic aldoxime dehydratase is mutated.

16. The method of claim 13, wherein conditions suitable for the formation of glucodiamine comprise hydrogen and a hydrogenation catalyst.

17. The method of claim 16, wherein the hydrogenation catalyst comprises at least one transition metal and a catalyst support.

18. The method of claim 13, wherein the hydrogenation catalyst is a supported bimetallic catalyst.

19. A chemoenzymatic method of producing a bio-based amide platform chemical, comprising:
contacting glucose with a biocatalyst under conditions suitable for the formation of glucodialdose;
contacting glucodialdose with a base under conditions suitable for the formation of glucodioxime; and
contacting glucodioxime with a hydrogenation catalyst in the presence of hydrogen under conditions suitable for formation of glucodiamine.

20. The method of claim 19, wherein the biocatalyst comprises a mutated galactose oxidase.

21. The method of claim 19, wherein the hydrogenation catalyst comprises a metal.

22. A method of preparing glucodiamine, comprising:
contacting glucose with an enzyme oxidation catalyst under conditions suitable for the formation of glucodialdose;
contacting glucodialdose with a nitrogen-containing compound under conditions suitable for the formation of an intermediate; and
reducing the intermediate under conditions suitable for the formation of glucodiamine.

23. The method of claim 1, wherein the enzyme oxidation catalyst comprises a copper radical oxidase.

24. The method of claim 1, wherein conditions suitable for the formation of glucodialdose comprise a temperature of from about 1° C. to about 70° C. and a pH of from about 5 to about 10.

25. The method of claim 1, wherein conditions suitable for the formation of glucodialdose further comprise the presence of a catalase, a small molecule activator, a single electron oxidizer, or combinations thereof.
